(19) United States
(12) Patent Application Publication
(10) Pub. No.: US 2026/0346006 A1
(43) Pub. Date: Nov. 5, 2026

(54) METHOD AND DEVICE FOR PROVIDING ELECTRODE ARRAY POSITION AND DIRECTION INFORMATION

(71) Applicant: FIELDCURE CO., LTD., Seoul (KR)

(72) Inventors: Jong Hyun KIM, Seoul (KR); Sang Min PARK, Seoul (KR)

(73) Assignee: FIELDCURE CO., LTD., Seoul (KR)

(21) Appl. No.: 19/111,818

(22) PCT Filed: Sep. 14, 2023
(86) PCT No.: PCT/KR2023/013787

§ 371 Date: Mar. 14, 2025

(30) Foreign Application Priority Data
Sep. 16, 2022 (KR) 10-2022-0116951
Sep. 8, 2023 (KR) 10-2023-0119706

(51) Int. Cl. A61N 1/36; A61B 90/00; A61N 1/04

(52) U.S. Cl. CPC A61N 1/3603; A61B 90/39; A61N 1/0476; A61B 2090/395

(57) ABSTRACT

A method for providing electrode array position and direction information according to an embodiment of the present invention is a method for providing electrode array position and direction information for electrical stimulation treatment of a subject, comprising the steps of: mounting, on a surface of the subject, a first display marker for displaying a center position and a reference direction of an electrode array; attaching an image marker to the surface of the subject so as to match the center position and the reference direction of the first display marker; capturing a medical image of the subject to which the image marker has been attached; providing the captured medical image to an external treatment planning system; receiving, from the external treatment planning system, a proposed position and a proposed direction of the electrode array on the basis of the image marker; and by reflecting the proposed position and tlie proposed direction with respect to the image marker, mounting a second display marker on the surface of the subject to match the proposed position and the pro-posed direction on the surface of the subject, and accordingly, the electrode array can be attached at an exact position and angle on an actual patient according to a treatment plan.

### CROSS-REFERENCE TO RELATED APPLICATION

This application is an International Application No. PCT/KR2023/013787 filed on Sep. 14, 2023, which is based on and claims the benefit of a Korean Patent Application No. 10-2023-0119706 filed on Sep. 8, 2023, in the Korean Intellectual Property Office, and of a Korean Provisional Patent Application No. 10-2022-0116951 filed on Sep. 16, 2022, in the Korean Intellectual Property Office, the disclosure of each of which is herein incorporated by reference in its entirety.

### TECHNICAL FIELD

The present disclosure relates to a method and an apparatus for providing position and direction information of an electrode array, and more particularly, a method and apparatus for providing position and direction information of an electrode array capable of attaching an electrode array including at least one electrode to a precise position and angle.

### BACKGROUND ART

The electrical stimulation therapies such as tDCS(Transcranial Direct Current Stimulation), TMS(Transcranial Magnetic Stimulation), TTFields(Tumor Treating Fields) and Hyperthermia are non-invasive treatments that are widely used to treat a variety of diseases depending on the location and intensity of the stimulation. The tDCS, TMS, TTFields, and Hyperthermia treatments can be used to create optimal treatment plans specific to the patient's individual characteristics through the treatment planning system software. However, there is a problem that it is difficult for doctors to attach an electrode array at the correct position and angle because there is no means to accurately map the position and angle of the electrode array determined in the treatment planning system to the actual position and angle attached to the patient.

Therefore, a method is needed for attaching the electrode array accurately according to the treatment plan while considering the position and angle of the electrode array simultaneously in order to increase the treatment effect, increase safety and reduce the risk.

### DISCLOSURE OF INVENTION

### Technical Problem

In order to solve the above, the present disclosure provides a method and apparatus for providing a position and direction of an electrode array capable of attaching an electrode array to an accurate position and direction.

### Solution to Problem

In order to solve the aforementioned object, the method of providing a position and direction information of an electrode array according to an embodiment of the invention concept is a method of providing a position and direction information of an electrode array for electrical stimulation therapy of a subject, the method may include mounting a first indicating marker on the surface of the subject to indicate a center position and a reference direction of the electrode array; attaching an image marker to the surface of the subject in alignment with the center position and the reference direction of the first indicating marker; recording a medical image of the subject on which the image marker is affixed; providing the recorded medical images to an external treatment planning system; receiving from the external treatment planning system a proposed position and a proposed direction of the electrode array based on the image marker; and mounting a second indicating marker on the surface of the subject in alignment with the proposed position and proposed direction derived from the image marker.

Here, the electrode array may include at least one individual electrode.

Here, the first indicating marker may be used to indicate the reference position and direction of the electrode array used in the electrical stimulation therapy.

Here, the inventive concept may further include mounting a third boundary indicating marker on the surface of the subject marking the boundary for the subject surface area that should not be used for the electrical stimulation therapy.

Here, the first indicating marker may be equal to or greater than the number of the image marker.

Here, the first and second indicating markers may be formed by at least one of methods of Temporary Tattoo, Henna, Pigment, Sticker and Skin Marker.

Here, the medical image may include at least one of CT, MRI, PET, SPECT and FMRI (Functional MRI), and the image marker may be formed of a marker that can be read in the medical image.

Here, the electrical stimulation therapy may be at least one of Tumor Treating Fields, Transcranial Direct Current Stimulation, Transcranial Alternating Current Stimulation, Transcranial Magnetic Stimulation and Radio-frequency Hyperthermia.

Here, the method of aligning with the proposed position and the proposed direction on the surface of the subject may involve following the projection line projected onto the surface of the subject.

Here, the method of aligning the proposed position and the proposed direction to the surface of the subject may be to follow the projection line projected onto the surface of the subject or identify the position and direction of the second indicating marker by performing translation and rotation from the first indicating marker using a laser display device using a patient-based coordinate system outside the subject.

In order to solve aforementioned object, a method of providing a position and direction information of an electrode array according to another embodiment of the invention concept is a method of providing a position and direction information of an electrode array for electrical stimulation therapy of the subject, the method may include mounting a plurality of first indicating markers for indicating a center position and a reference direction of the electrode array on the surface of a subject at an predetermined intervals; attaching a plurality of image markers to the surface of the subject in alignment with the center position and the reference direction of the plurality of first indicating markers; recording a medical image of the subject to which the plurality of image markers is attached; providing the recorded medical image to an external treatment planning system; receiving a proposed position and a proposed direction of the electrode array based on the image marker from the external treatment planning system; and based on the proposed position and the proposed direction derived from the image marker, mounting a second indicating marker on the surface of the subject in alignment with the proposed position and the proposed direction.

In order to solve the aforementioned object, an apparatus for providing a position and a direction information of an electrode array according to an embodiment of the invention is an apparatus 1000 for providing a position and a direction information of an electrode array for electrical stimulation therapy of a subject, the apparatus 1000 may include an indicating marker unit 1100 mounting a first indicating marker for indicating a center position and a reference direction of the electrode array on a surface of a subject; an image marker unit 1200 attaching an image marker to the surface of the subject in alignment with the center position and the reference direction of the first indicating marker; a proposed position and direction determination unit 1300 recording a medical image of the subject to which the image marker is attached, providing the recorded medical image to an external treatment planning system and receiving a proposed position and a proposed direction of the electrode array based on the image marker from the external treatment planning system. The indicating marker unit 1100 may mount a second indicating marker 1140 on the surface of the subject in alignment with the proposed position and proposed direction on the surface of the subject.

Here, the electrode array may include at least one individual electrode.

Here, the first indicating marker 1120 may be used to indicate the reference position and direction of the electrode array used in the electrical stimulation therapy.

Here, the indicating marker unit 1100 may mount a third boundary indicating marker 1160 on the surface of the subject marking the boundary for the subject surface area that should not be used for the electrical stimulation therapy.

Here, the first indicating marker 1120 may be equal to or greater than the number of the image marker 1220.

Here, the first marker 1120 and the second marker 1140 may be formed by at least one of the methods of Temporary Tattoo, Henna, Pigment, Sticker and Skin Marker.

Here, the medical image may include at least one of CT, MRI, PET, SPECT and FMRI (Functional MRI), the image marker 1220 may be formed of a marker that can be read in the medical image.

Here, the electrical stimulation therapy may be at least one of Tumor Treating Fields, Transcranial Direct Current Stimulation, Transcranial Alternating Current Stimulation, Transcranial Magnetic Stimulation and Radio-frequency Hyperthermia.

Here, the method of mounting the second indicating marker 1140 on the surface of the subject in alignment with the proposed position and the proposed direction based on the image marker provided by the proposed position and direction determination unit 1300 may be to follow the projection line projected onto the subject surface.

Here, the method of mounting the second indicating marker 1140 on the surface of the subject in alignment with the proposed position and the proposed direction based on the image marker provided from the proposed position and direction determination unit 1300 may be to follow the projection line projected onto the subject surface, or identify the position and the direction of the second indicating marker 1140 by performing translation and rotation from the first indicating marker 1120 using a laser display device using a patient-based coordinate system outside the subject.

In order to solve the aforementioned object, an apparatus of providing a position and a direction information of an electrode array according to an embodiment of the invention concept is an apparatus 1000 for providing a position and a direction information of an electrode array for electrical stimulation therapy of a subject, the apparatus 1000 may include an indicating marker unit 1100 mounting a plurality of first indicating markers for indicating a center position and a reference direction of the electrode array on the surface of the subject at predetermined interval; an image marker unit 1200 attaching a plurality of image markers to the surface of the subject in alignment with the center position and the reference direction of the plurality of first indicating markers; a proposed position and direction determination unit 1300 recording a medical image of the subject to which the plurality of image markers is attached, providing the recorded medical image to an external treatment planning system and receiving a proposed position and a proposed direction of the electrode array based on the plurality of image markers from the external treatment planning system. The indicating marker unit 1100 may mount a second indicating marker 1140 on the surface of the subject in alignment with the proposed position and the proposed direction based on the plurality of image markers provided from the proposed position and direction determination unit 1300.

### Advantageous Effects of Invention

According to the inventive concept, a positional directional indicator-based stereotactic device providing information about the center coordinates and angle of the electrodes is used and medical images taken for each subject are acquired, and the position and the angle information of the electrode array for the subject is derived based on them, so that the position and the angle of the electrode array according to the treatment plan can be attached to the exact position and angle of the actual patient.

In addition, not only the coordinates of the electrode but also the angle of the electrode array can be indicated by using a positional directional indicator so that more precise position and direction of the electrode array can be utilized. The method and system of providing the position and direction information of the electrode array using such a stereotactic device can improve the accuracy of the procedure during electrical stimulation therapy, and the electrode can be attached visually and intuitively.

According to the inventive concept, by using a method and system for providing position and angle information of an electrode array using a stereotactic device, the optimal stimulus position and direction can be shown on a coordinate system, so that the patient or guardian as well as the medical staff can effectively identify the position to attach the electrode to an accurate position.

### BRIEF DESCRIPTION OF DRAWINGS

FIG. 1 is a view illustrating an apparatus for providing a position and a direction information of an electrode array according to an embodiment of the inventive concept.

FIG. 2 is a view illustrating a method for providing a position and a direction information of an electrode array according to another embodiment of the inventive concept.

FIG. 3 is a diagram illustrating a method for providing a position and a direction information of an electrode array according to another embodiment of the inventive concept.

FIG. 4 is a view illustrating problems of a conventional arts.

FIGS. 5A and 5B are a view illustrating a marker in a method for providing a position and a direction information of an electrode array according to another embodiment of the inventive concept.

FIGS. 6A and 6B are a view illustrating a method of attaching a marker in a method for providing a position and a direction information of an electrode array according to another embodiment of the inventive concept.

FIGS. 7A and 7B are a view illustrating that an indicating marker and an image marker is identified in a method for providing a position and a direction information of an electrode array according to another embodiment of the inventive concept.

FIGS. 8A and 8B are a view illustrating a method of identifying a coordinate of a position after determining the position of an electrode array in a method for providing a position and a direction information of an electrode array according to another embodiment of the inventive concept.

FIGS. 9A and 9B are a view illustrating a selection of one electrode array based on a position and an angle information of output electrode array in a method for providing a position and a direction information of an electrode array according to another embodiment of the inventive concept.

FIG. 10 is a view illustrating that a marker can be attached at various angles in a method for providing a position and a direction information of an electrode array according to another embodiment of the inventive concept.

FIG. 11 is a view illustrating a position and angle where the electrode array should be inhibited from being attached in a method for providing a position and a direction information of the electrode array according to another embodiment of the inventive concept.

FIG. 12 is a view illustrating a variety of generating a marker in a method for providing a position and a direction information of an electrode array according to another embodiment of the inventive concept.

FIG. 13 shows a wearable device in the form of a vest 800 and a bellyband 900 in which markers are pre-arranged in the method of providing position and a direction information of an electrode array according to another embodiment of the inventive concept.

### MODE FOR THE INVENTION

Embodiments of the inventive concept will be described more fully hereinafter with reference to the accompanying drawings and let those skilled in the art implement easily. It should be noted, however, that the inventive concept is not limited to the following embodiments and may be implemented in various forms.

For describing the inventive concept obviously, other shape not related to description are excluded and the same reference designators denote the same or similar elements throughout the specification.

If there is referred that a part is connected to another part, it includes a case of ‘connecting directly’ with each other by interposing another device as well as a case of ‘connecting electrically’ with each other.

In addition, if there is referred that any part ‘include(s)’ a component, it means that other components are not excluded but further includes another component unless special opposite statement is described.

If there is referred that a part ‘on’ another part, it is on the part or another part may be interposed therebetween. In contrast, if there is referred that a part ‘directly on’ another part, any part is not interposed therebetween.

Terms of a first, a second, a third will be used to describe various parts, elements, regions, layers and/or sections, but not limited to these. These terms are used only to distinguish a part, element, region, layer or section with another part, element, region, layer or section. Therefore, a first part, element, region, layer or section may be described hereinafter a second part, element, region, layer or section within a scope of the inventive concept.

The professional terms will be used hereinafter in order to mention only specific embodiment, but it does not intend to limit the inventive concept. The wording in the singular used here will include the plural unless obvious opposite meaning is expressed. The meaning of ‘include’ used in this description will embody curtain feature, region, integer number, step, action, component and/or element, but not exclude exist or supplement of other feature, region, integer, step, action, component and/or element.

Terms of opposite spaces such as ‘up’ and ‘down’ may be used in order to easily describe a relationship between a part and another part illustrated in a drawing. These terms will be intended to include another meaning or operation of utilized device with the meaning intended in drawings.

For example, when the device on the drawing is turned over, a part described as being ‘under’ other parts are described as being ‘on’ the other parts. So, illustrative term ‘under’ includes both directions of up and down. The device may be rotated 90 degrees or other angles, and terms denoting relative space are interpreted accordingly.

All terms including technical terms and scientific terms used herein, even if not defined differently, have the same meaning as commonly understood by those skilled in the art of the inventive concept. Terms defined in commonly used dictionaries are additionally interpreted as having meanings correspondent with related technical documents and current described content, and not interpreted in ideal or very formal meanings unless defined.

An embodiment will now be described more fully hereinafter with reference to the accompanying drawings to facilitate implementation of the inventive concept by those skilled in the art. However, the inventive concept may be implemented in various forms and not limited to the following some embodiments.

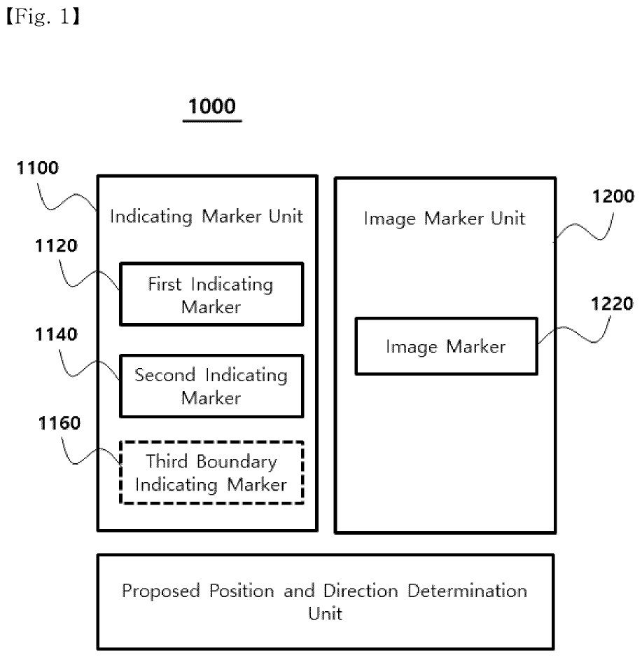

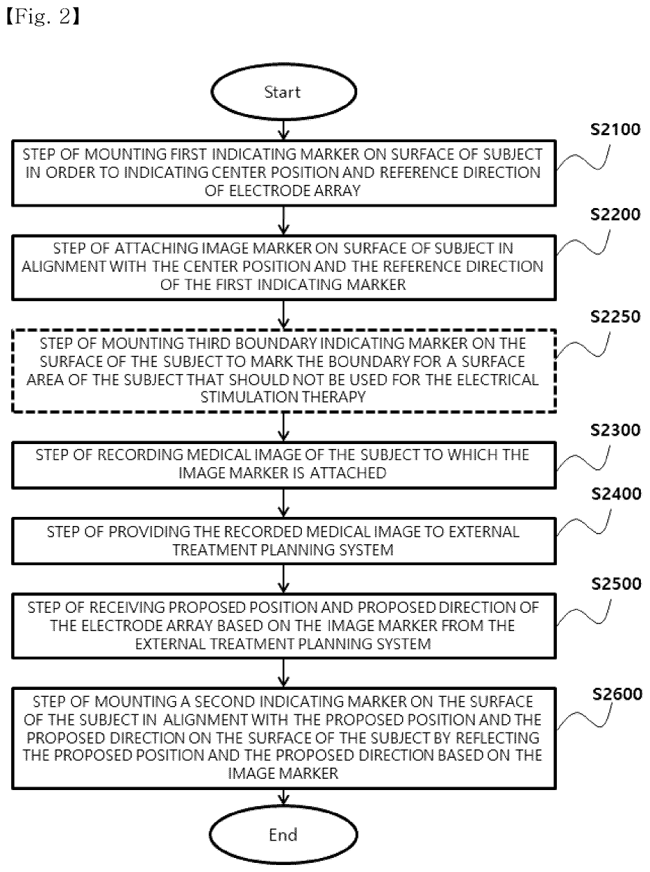

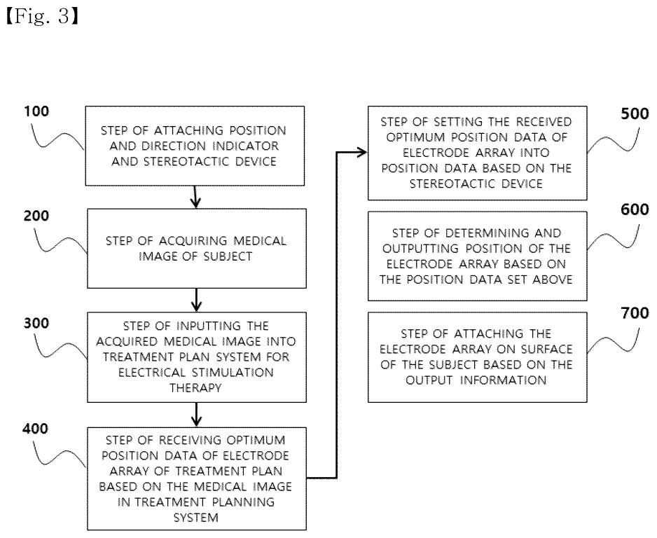

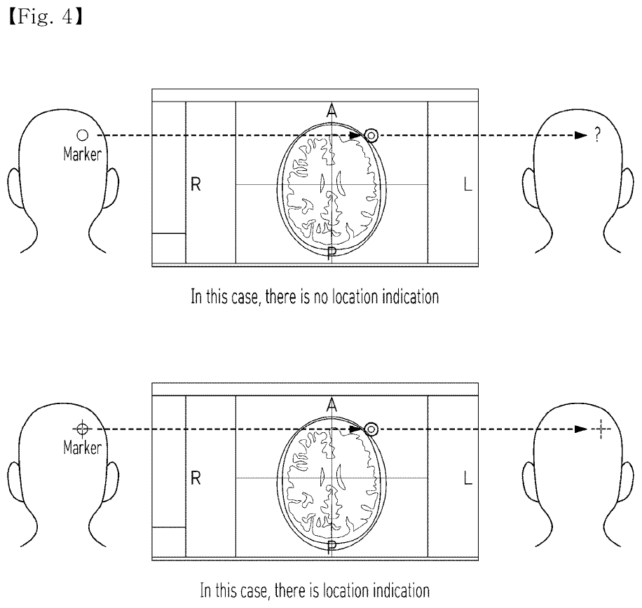

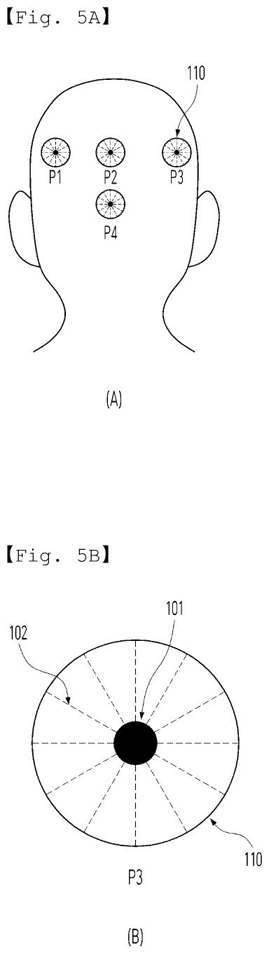

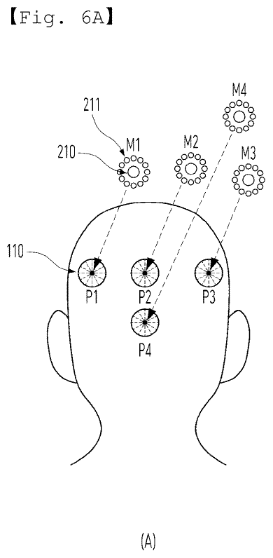

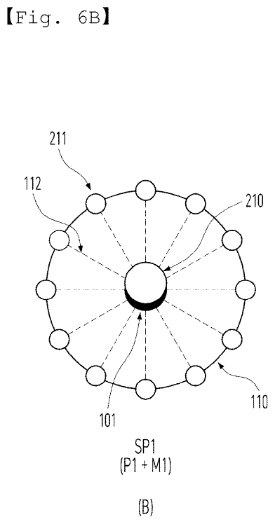

FIG. 1 is a view illustrating an apparatus for providing a position and direction information of an electrode array according to an embodiment of the inventive concept. FIG. 2 is a view illustrating a method for providing a position and direction information of an electrode array according to another embodiment of the inventive concept. FIG. 3 is a diagram illustrating a method for providing a position and direction information of an electrode array according to another embodiment of the inventive concept. FIG. 4 is a view illustrating problems of a conventional arts. FIG. 5 is a view illustrating a marker in a method for providing a position and direction information of an electrode array according to another embodiment of the inventive concept. FIG. 6 is a view illustrating a method of attaching a marker in a method for providing a position and direction information of an electrode array according to another embodiment of the inventive concept.

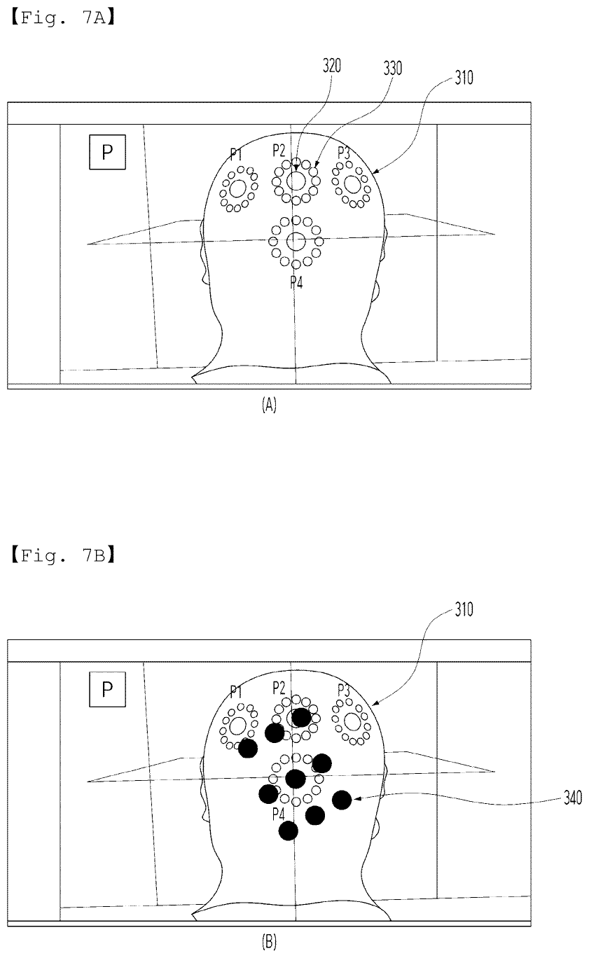

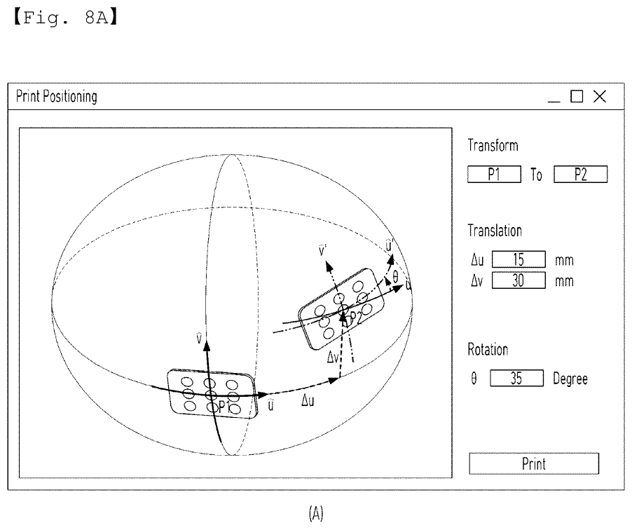

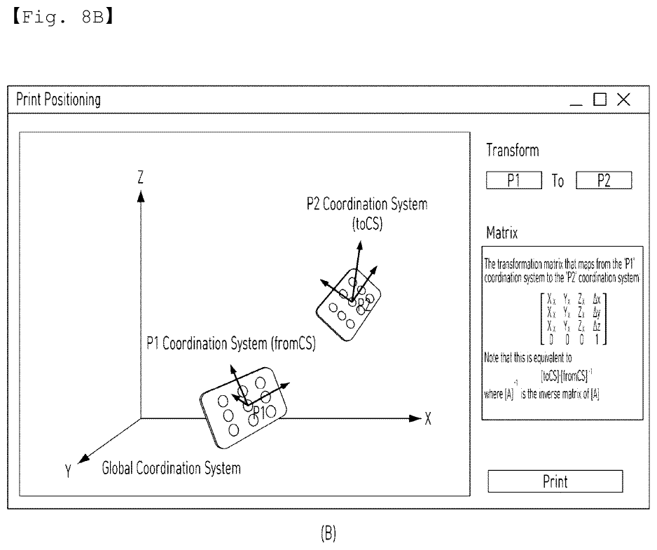

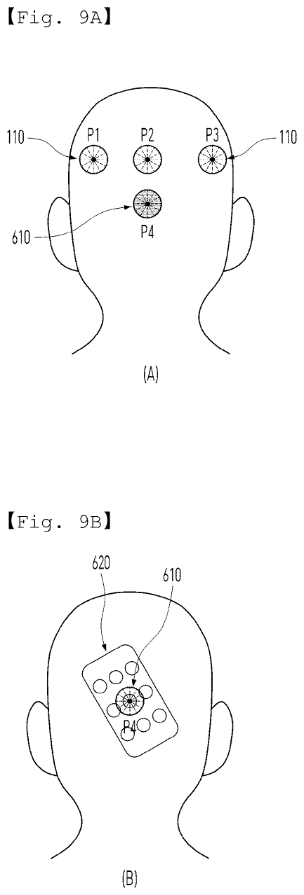

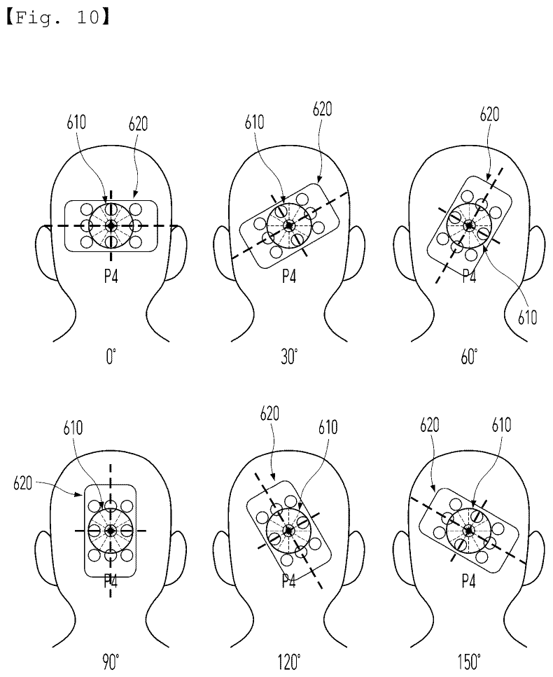

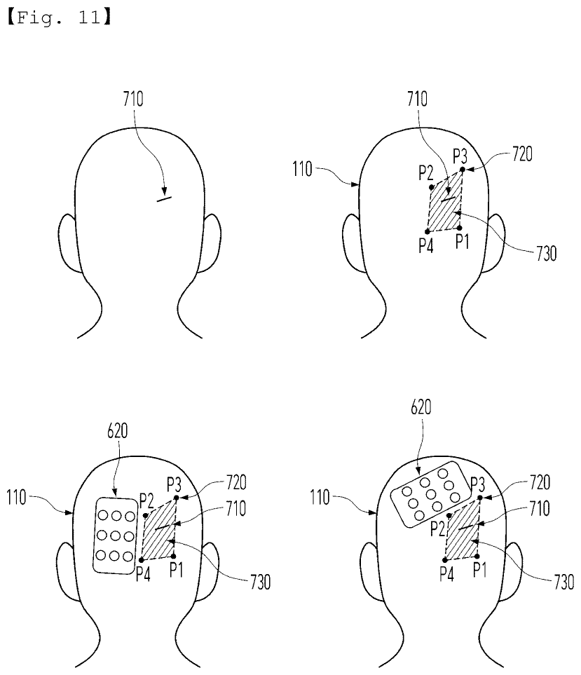

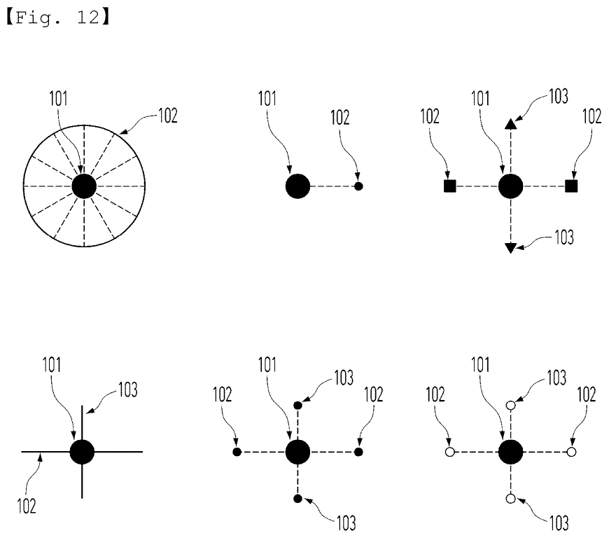

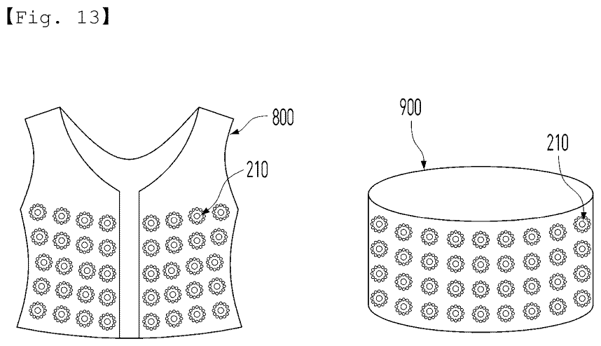

FIG. 7 is a view illustrating that an indicating marker and an image marker is identified in a method for providing a position and direction information of an electrode array according to another embodiment of the inventive concept. FIG. 8 is a view illustrating a method of identifying a coordinate of a position after determining the position of the electrode array in a method for providing a position and direction information of the electrode array according to another embodiment of the inventive concept. FIG. 9 is a view illustrating a selection of one electrode array based on a position and an angle information of output electrode array in a method for providing a position and direction information of an electrode array according to another embodiment of the inventive concept. FIG. 10 is a view illustrating that a marker can be attached at various angles in a method for providing position and direction information of an electrode array according to another embodiment of the inventive concept. FIG. 11 is a view illustrating a position and angle where the electrode array should be inhibited from being attached in a method for providing a position and direction information of the electrode array according to another embodiment of the inventive concept. FIG. 12 is a view showing illustrating a variety of generating a marker in a method for providing a position and direction information of an electrode array according to another embodiment of the inventive concept. FIG. 13 shows a wearable device in the form of a vest 800 and a bellyband 900 in which markers are pre-arranged in the method of providing position and direction information of an electrode array according to another embodiment of the inventive concept.

Referring to FIG. 2, the method of providing position and direction information of an electrode array according to an embodiment of the invention concept is a method for electrical stimulation therapy of the subject. The method may include: mounting a first indicating marker for indicating a center position and a reference direction of an electrode array on the surface of the subject S2100; attaching an image marker to the surface of the subject in alignment with the center position and the reference direction of the first indicating marker S2200; recording a medical image of the subject to which the image marker is attached S2300; providing the recorded medical image to an external treatment planning system S2400; receiving a proposed position and a proposed direction of the electrode array based on the image marker from the external treatment planning system S2500; and based on the proposed position and the proposed direction derived from the image marker, mounting a second indicating marker on the surface of the subject in alignment with the proposed position and the proposed direction (S2600).

Here, the electrode array may include at least one individual electrode.

In the step S2100 of mounting the first indicating marker on the surface of the subject, the first indicating marker may be intended to indicate the center position and the reference direction of the electrode array, and may be used to indicate the reference position and direction of the electrode array used for the electrical stimulation therapy.

The step S2200 of attaching the image marker to the surface of the subject may attach the image marker to the surface of the subject in alignment with the center position and the reference direction of the first indicating marker.

On the other hand, the method may further include mounting a third boundary indicating marker on the surface of the subject to indicate a boundary for the subject surface area that should not be used in the electrical stimulation therapy S2250.

The step S2250 of mounting the third boundary indicating marker on the surface of the subject may indicate the boundary to the surface area of the subject which should not be used in the electrical stimulation therapy, and may indicate the boundary to the area where it is not suitable to attach the electrode array for electrical stimulation due to the presence of skin rash, implantable medical device and other electrodes on the surface of the subject.

In the step S2200 of attaching the image marker to the surface of the subject, the first indicating marker may be equal to or more than the number of the image markers. For example, the first indicating marker may be used to indicate all the positions where the image marker can be attached, and the image marker may be attached by selecting only the optimal position in all the attachable positions.

The step S2300 of recording a medical image of the subject may record a medical image with the image marker attached. Since the image marker can be identified in the medical image, it will be possible to obtain a medical image that reflects the position of the image marker.

The step S2400 of providing the recorded medical image to the external treatment planning system and the step S2500 of receiving the proposed position and the proposed direction from the external treatment planning system comprise providing the medical image that shows the position of the image marker to the external treatment planning system, and receiving the proposed position and the proposed direction based on the image marker, that is, the optimal electrode array position and direction from the external treatment planning system.

The step S2600 of mounting the second indicating marker on the surface of the subject in alignment with the proposed position and the proposed direction comprises mounting the second indicating marker based on the position and the direction information derived from the image marker. In other words, the second indicating marker enables mounting an electrode array on the surface of the subject in accordance with the optimal position and direction provided by the external treatment planning system for subsequent treatment.

In addition, the first and the second indicating markers may be formed by at least one method of Temporary Tattoo, Henna, Pigment, Sticker and Skin Marker. The first indicating marker may be temporarily attached because it can only be maintained for a certain period of time, but the second indicating marker is used continuously during subsequent treatment, so it will be recommended to be able to maintain the attachment during the treatment process.

On the other hand, the medical image may include at least one of CT, MRI, PET, SPECT and FMRI (Functional MRI), and the image marker may be formed of a marker that can be read in the medical image.

In addition, the electrical stimulation therapy may be at least one of Tumor Treating Fields, Transcranial Direct Current Stimulation, Transcranial Alternating Current Stimulation, Transcranial Magnetic Stimulation and Radio-frequency Hyperthermia.

In particular, the method of aligning the proposed position and the proposed direction on the surface of the subject may involve following the projection line projected onto the surface of the subject. Since the surface of the subject is not usually flat, it is reasonable to attach a second indicating marker to the surface of the subject along the projection line in order to properly align with the proposed position and the proposed direction.

The method of aligning the proposed position and the proposed direction on the surface of the subject may be to identify the position and direction of the second indicating marker by following the projection line projected on the surface of the subject, or by performing translation and rotation from the first indicating marker using a laser display device with a patient-based coordinate system outside the subject.

The method of providing position and direction information of an electrode array according to another embodiment of the invention concept is a method of providing position and direction information of an electrode array for electrical stimulation therapy of the subject, the method may include mounting a plurality of first indicating markers for indicating a center position and a reference direction of the electrode array on the surface of the subject at an predetermined intervals; attaching a plurality of image markers to the surface of the subject in alignment with the center position and the reference direction of the plurality of first indicating markers; recording a medical image of the subject to which the plurality of image markers is attached; providing the recorded medical image to an external treatment planning system; receiving a proposed position and a proposed direction of the electrode array based on the image marker from the external treatment planning system; and based on the proposed position and the proposed direction derived from the image marker, mounting a second indicating marker on the surface of the subject in alignment with the proposed position and the proposed direction.

Referring to FIG. 1, the apparatus of providing position and direction information of an electrode array according to an embodiment of the inventive concept is an apparatus 1000 of providing position and direction information of an electrode array for electrical stimulation therapy of the subject, the apparatus 1000 may include an indicating marker unit 1100 mounting a first indicating marker 1120 for indicating a center position and a reference direction of an electrode array on the surface of the subject; an image marker unit 1200 attaching the image marker 1220 to the surface of the subject in alignment with the center position and the reference direction of the first indicating marker 1120; a proposed position and direction determination unit 1300 recording a medical image of the subject to which the image marker 1220 is attached, providing the recorded medical image to an external treatment planning system, and receiving a proposed position and a proposed direction of the electrode array based on the image marker from the external treatment planning system. The indicating marker unit 1100 may mount a second indicating marker 1140 on the surface of the subject in alignment with the proposed position and the proposed direction based on the image marker 1220.

Here, the electrode array may include at least one individual electrode.

The indicating marker unit 1100 may mount the first indicating marker 1120 for indicating the center position and the reference direction of the electrode array on the surface of the subject. In particular, the first indicating marker 1120 may indicate the reference position and direction of the electrode array to be used for the electrical stimulation therapy.

On the other hand, the indicating marker unit 1100 may further include a third boundary marker 1160 indicating the boundary for the subject surface area, and the third boundary marker 1160 may indicate the boundary for the subject surface region that should not be used for the electrical stimulation therapy.

Consequently, the third boundary marker 1160 may be further mounted on the surface of the subject.

The image marker unit 1200 may further attach the image marker 1220 to the surface of the subject in alignment with the center position and reference direction of the first indicating marker 1120. In particular, the first indicating marker 1120 may be equal to or greater than the number of the image marker 1220. For example, the first indicating marker 1120 may be used to indicate all the positions to which the image marker 1220 can be attached, and the image marker 1220 can be attached by selecting only the optimal position in all the attachable positions.

The proposed position direction determination unit 1300 may record a medical image for the subject where the image marker 1220 is attached, provide the recorded medical image to the external treatment planning system, and receive the proposed position and the proposed direction based on the image marker 1220 from the external treatment planning system.

On the other hand, the indicating marker unit 1100 may mount the second indicating marker 1140 on the surface of the subject in alignment with the proposed position and proposed direction based on the image marker 1220 provided by the proposed position direction determination unit 1300.

Here, the first indicating marker 1120 and the second indicating marker 1140 may be formed by at least one method of Temporary Tattoo, Henna, Pigment, Sticker and Skin Marker. The first indicating marker may be temporarily attached because it can be maintained just for a certain period of time, but the second indicating marker is to be used continuously during subsequent treatment, so it will be recommended to be able to maintain the attachment during the treatment process.

On the other hand, the indicating marker unit 1100 may include a third boundary indicating marker 1160 on the surface of the subject indicating the boundary to the surface area of the subject which should not be used in electrical stimulation

The third boundary marker 1160 may indicate the boundary to the surface area of the subject which should not be used in the electrical stimulation therapy, and may indicate the boundary to the area where it is not suitable to attach the electrode array for electrical stimulation due to skin rash on the surface of the subject, the presence of implantable medical devices, other electrodes.

On the other hand, the medical image may include at least one of CT, MRI, PET, SPECT and FMRI (Functional MRI), and the image marker may be formed of a marker that can be read in the medical image.

In addition, the electrical stimulation therapy may be at least one of Tumor Treating Fields, Transcranial Direct Current Stimulation, Transcranial Alternating Current Stimulation, Transcranial Magnetic Stimulation and Radio-frequency Hyperthermia.

The method of mounting the second indicating marker 1140 on the surface of the subject in alignment with the proposed position and the proposed direction based on the image marker provided by the proposed position direction determination unit 1300 may mount along the projection line projected onto the subject surface. Since the surface of the subject is not usually flat, it would be reasonable to attach a marker to the surface of the subject along the projection line in order to align with the proposed position and the proposed direction on the surface of the subject.

The method of mounting the second indicating marker 1140 on the surface of the subject in alignment with the proposed position and the proposed direction based on the image marker 1220 provided from the proposed position direction determination unit 1300 may identify the position and the direction of the second indicating marker 1140 by following the projection line projected onto the subject surface or by performing translation and rotation from the first indicating marker 1120 using a laser display device using a patient-based coordinate system outside the subject.

The apparatus of providing position and direction information of an electrode array according to one embodiment of the inventive concept is an apparatus 1000 of providing position and direction information of an electrode array for electrical stimulation therapy of the subject, the apparatus 1000 may include an indicating marker unit 1100 mounting a plurality of first indicating markers for indicating a center position and a reference direction of the electrode array on a surface of a subject at predetermined intervals; an image marker unit 1200 attaching a plurality of image markers to the surface of the subject in alignment with the center position and the reference direction of the plurality of first indicating markers; a proposed position direction determination unit 1300 recording a medical image of the subject to which the plurality of image markers is attached, providing the recorded medical image to an external treatment planning system, and receiving a proposed position and a proposed direction of the electrode array based on the plurality of image markers from the external treatment planning system. The indicating marker unit 1100 may mount a second indicating marker 1140 on the surface of the subject in alignment with the proposed position and the proposed direction based on the plurality of image markers provided from the proposed position direction determination unit 1300.

In general, an electrical stimulation system performs treatment by attaching an electrode array containing at least one individual electrode directly on the human body to form an electromagnetic field inside the human body. In this case, various treatment plans may be generated depending on the position and angle of the attached electrode array, and an optimal treatment plan for the patient is generated through electrical stimulation simulation based on the patient's medical image using the treatment planning system.

In order to determine the optimal treatment plan, it is necessary to find the position and angle of the electrode array that provides maximum electrical stimulation to the target and minimal stimulation outside the target. However, after acquiring the optimal position and angle information of the electrode array through the simulation using the treatment planning system, it is necessary to attach the electrode array to the actual patient based on the position and direction information obtained above, but there are several difficulties.

It is very difficult to align the coordinates and angles of the electrode arrays in the treatment planning system, such as the position and the direction of the electrode arrays, with the coordinates and angles of actual electrical stimulation arrays on the patient.

Thus, in the embodiment of the inventive concept described hereinafter, a position direction indicator may be marked on the subject, and the position and direction marker may be attached to the position direction indicator and identified in a medical image so that the treatment planning system can be aligned with the actual patient's reference coordinates and angles. The ‘position and the direction of the electrode array based on the position direction indicator’ is output and the electrode array may be attached on the surface of the subject.

Referring to FIG. 3, hereinafter, the method and apparatus for providing position and direction information of an electrode array according to an embodiment of the inventive concept are described in more detail.

FIG. 3 is a diagram illustrating a method for providing a position and direction information of an electrode array according to another embodiment of the inventive concept.

Referring to FIG. 3, the method and the apparatus for providing position and direction information of an electrode array according to an embodiment of the inventive concept may mark a position direction indicator on the surface of a subject. The marked position direction indicator may be a marker placed on the subject, and the type of the marker may be a temporary tattoo, skin dyeing (henna), pigment injection (pigment), sticker (Sticker), marking pen (skin marker), etc. As methods to draw the direction indicator, there are a direct measurement using a natural marker of the human body (Nasion, Inion, right ear, left ear) and an indirect measurement using laser. A stereotactic device that corresponds 1:1 to the generated position direction indicator may be attached. The stereotactic device may be used to identify in the medical image, CT, MRI, SPECT, Ultra Sound, etc. may be used as the medical image, and a substance used in the stereotactic device for displaying in the medical image may be a marker that can be identified in medical images such as MR-marker, CT-marker, etc.

In a subject and stereotactic device image acquisition unit, medical image may be acquired for using an electrical stimulation simulation. The medical image that can identify the subject and the stereotactic device attached to the subject is acquired, and the optimal electrical stimulation treatment plan may be generated through the step 300 of inputting the acquired medical image into the treatment plan system, and the position of the electrode array may be received through the step 400 of receiving the electrode array position data of the generated treatment plan. At this time, a position data attachable on a subject body may be set based on the stereotactic device 500. In the step 600 of determining and outputting the position and direction of the electrode array based on the position data set above, the position and the direction may be output to the user. The array position and direction information may be output by selecting one of several stereotactic devices set on the target surface, and the position coordinates and angular direction may be set based on the selected stereotactic device. Through the above, the angle of the electrode array may be output such that the treatment plan can be applied with considering the direction. In the step 700 of attaching the electrode array to the surface of the subject body based on the output information, it is a step of attaching directly to the patient based on the output position coordinates and the angular direction information, and the position and the direction of the electrode array can be aligned through the position direction indicators marked on the actual patient and output from the stereotactic device.

FIG. 4 is a view illustrating problems of a conventional arts. If a medical image is acquired after attaching an image marker that provides position information on the human body, the position of the marker can be identified in the medical image, but it is difficult to accurately identify a position of the marker on the patient after acquiring the position of the marker in the medical image. In order to solve this problem, a marker should be attached to the patient's skin along with an image marker.

FIGS. 5A and 5B are a view illustrating a marker in a method for providing a position and direction information of an electrode array according to another embodiment of the inventive concept. A position direction indicator 110 is shown to solve this technique, and the position direction indicator may be divided into a coordinate indicator 101 indicating coordinates of a center and an angle indicator 102 indicating the direction. By attaching the position direction indicator, it can serve as a reference point for the electrode array to be attached to the subject surface. A position direction indicator is information that allows the patient or specialist to refer and to attach the position and direction of the stimulator to the optimal point of stimulation in the optimal direction.

FIGS. 6A and 6B are a view illustrating a method of attaching a marker in a method for providing a position and direction information of an electrode array according to another embodiment of the inventive concept. It is a method of attaching a stereotactic device on the generated position direction indicator, and the stereotactic device may be basically divided into two types of a coordinate marker that indicates the center position of the electrode array and an angle marker that represents the center angle of the electrode array. It may be attached on the center point of the electrode in order to identify the angle marker 211 based on the angle indicator 112 and a coordinate marker 210 based on the direction indicator 101, and may be attached in various other ways such as a cross net. At this time, in order to distinguish between the coordinate marker and the angle marker, the shape and size of each marker may be modified, i.e., the size of the coordinate marker and the angle marker may be different each other or the shape of the coordinate marker and the angle marker may be different each other. The stereotactic position (SP1) generated by the above may refer to the coordinates to which the stereotactic device is attached.

FIGS. 7A and 7B are a view illustrating that an indicating marker and an image marker is identified in a method for providing a position and direction information of an electrode array according to another embodiment of the inventive concept.

Identification of the coordinate marker and the angle marker in the treatment planning system is shown, and the patient and stereotactic device may be represented. In the treatment planning system, a 3D model may be created based on the medical image, and the coordinate marker 320 and angle marker 330 may be identified in the medical image, so that the coordinate and the angle markers may also be represented as a 3D model.

FIGS. 8A and 8B are a view illustrating a method of identifying a coordinate of a position from a first indicating marker P1 after determining the position, i.e., a second indicating marker P2 of the electrode array in a method for providing a position and direction information of the electrode array according to another embodiment of the inventive concept. This method may involve two approaches. Here, P1 and P2 represent a local coordinate system with origin at the center of the array.

First, it follows the projection line projected onto a surface of the subject as shown in FIG. 8A, which projects the two axes (u-axis and v-axis) of the two-dimensional coordinate system of the electrode array onto the subject, and expresses P2 by a distance and an angle from P1 along these axes so that P2 can be identified. As shown in FIG. 8A, P2 may be identified by translating Δu along the u-axis, Δv along the v-axis of P1 projected onto the surface of the subject and then rotating (θ).

Second, in a patient-based coordinate system, P2 may be identified as a transformation of P1. This method may identify the position and the direction of P2 by performing translation and rotation from P1 using a laser display device that uses a patient-based coordinate system outside the subject. These translation and rotation may be described as follows using the affine transformation matrix, as shown in FIG. 8B.

P2·P1⁻¹

Here, P1 and is represented by a 4×4 matrix representing each coordinate system, and P1⁻¹ is the inverse matrix of the P1.

FIGS. 9A and 9B are a view illustrating a selection of one electrode array based on a position and a direction information of output electrode array in a method for providing a position and direction information of an electrode array according to another embodiment of the inventive concept. In embodiments, FIG. 9A shows positional direction indicators P1, P2, P3, P4, and FIG. 9B shows attaching an electrode array 620 to the selected positional direction indicator P4 in consideration of the coordinates and angle of the center point.

FIG. 10 is a view illustrating that a marker can be attached at various angles in a method for providing a position and direction information of an electrode array according to another embodiment of the inventive concept. It shows that the marker may be attached at various angles when the electrode array 720 is attached to the position indicator 710. In this embodiment, the number of angles of the position indicator is expressed as 12, representing a total of 6 angles, but not limited thereto. The Angle can be expressed by aligning the center line with the angle indicator based on the center line of the electrode array.

FIG. 11 is a view illustrating a position and angle where the electrode array should be inhibited from being attached in a method for providing a position and direction information of the electrode array according to another embodiment of the inventive concept. There is a site 730 that cannot be attached due to various reasons in the human body, e.g., skin rash, implantable medical device or the presence of other electrodes, and a stereotactic device 720 may also be used to identify it in the treatment planning system. Stereotactic device 720 may be used to determine the position and angle of the site. For example, in this embodiment, four stereotactic devices were used to divide sectors 730. After identification in the treatment planning system, the position and angle of the electrode array 620 may be determined.

FIG. 12 is a view illustrating a variety of generating a marker in a method for providing a position and direction information of an electrode array according to another embodiment of the inventive concept. In this embodiment, various angles are described as a method A, but they can also be generated in various ways. Basically, the long axis 102 and the short axis 103 are displayed separately.

As methods for representing the minimum position and angle, there are methods that represents with at least two indicators B, takes different shapes of coordinate indicator and angle indicator C, represents the long axis and the short axis of the angle indicator as straight lines D, and makes strengths of the angle indicator different F. Using these various methods, the position indicator may be utilized by selecting the position indicator specific to an individual patient.

FIG. 13 shows a wearable device in the form of a vest 800 and a bellyband 900 in which indicators and markers are pre-arranged in the method of providing position and direction information of an electrode array according to another embodiment of the inventive concept.

When the target point of electrical stimulation therapy is located in the chest or abdomen, the vest type 800 or the bellyband type 900 may be worn, and the medical images are acquired. The position indicator indicating the position of the electrode array and the angle indicator may be pre-arranged on the vest type 800 or the belly band type 900. As position markers and angle markers can be identified in the medical images taken above, it is possible to create a treatment plan based on position markers and angle markers. By identifying the position of the electrode array used in the treatment plan created above, it is possible to attach the electrode array of the treatment plan using a wearable device in the form of a vest or belly band without marking a direction indicator on the subject.

While the inventive concept has been described with reference to accompanying drawings, it will be apparent to those skilled in the art that various changes and modifications may be made without departing from the spirit and scope of the inventive concept. For example, a person skilled in the art may change the material, size, etc. of each component according to the field of application, or combine or replace the embodiments disclosed and perform them in a form that is not clearly disclosed in the embodiment of the present invention, however, this does not depart from the scope of the present invention. Thus, the scope of the inventive concept is to be determined by the broadest permissible interpretation of the following claims and their equivalents.
1000: Apparatus for providing position and direction information of electrode array
1100: Indicating marker unit
1120: First indicating marker
1140: Second indicating marker
1160: Third boundary indicating marker
1200: Image marker unit
1220: Image marker
1300: Proposed position direction determination unit

1. A method for providing position and direction information of an electrode array for electrical stimulation therapy of a subject comprising:
mounting a first indicating marker for indicating a center position and a reference direction of the electrode array on the surface of the subject;
attaching an image marker to the surface of the subject in alignment with the center position and the reference direction of the first indicating marker;
acquiring a medical image of the subject on which the image marker is attached;
providing the acquired medical images to an external treatment planning system;
receiving a proposed position and a proposed direction of the electrode array based on the image marker from the external treatment planning system; and
mounting a second indicating marker on the surface of the subject in alignment with the proposed position and the proposed direction.

2. The method of claim 1, wherein the electrode array comprises at least one individual electrode.

3. The method of claim 1, further comprising:
mounting a third boundary indicating marker on the surface of the subject to mark the boundary for a surface area of the subject that should not be used for the electrical stimulation therapy.

4. The method of claim 1, wherein the number of first indicating markers is equal to or greater than the number of the image markers.

5. The method of claim 1, wherein the first and second indicating markers are formed by at least one method of Temporary Tattoo, Henna, Pigment, Sticker and Skin Marker.

6. The method of claim 1, wherein the medical image includes at least one of CT, MRI, PET, SPECT and FMRI (Functional MRI), and
the image marker is formed of a marker that can be read in the medical image.

7. The method of claim 1, wherein the electrical stimulation therapy is at least one of Tumor Treating Fields, Transcranial Direct Current Stimulation, Transcranial Alternating Current Stimulation, Transcranial Magnetic Stimulation and Radio-frequency Hyperthermia.

8. The method of claim 1, wherein aligning with the proposed position and proposed direction on the surface of the subject comprises following the projection line projected onto the surface of the subject.

9. The method of claim 8, wherein aligning with the proposed position and proposed direction on the surface of the subject further comprises performing translation and rotation from the first indicating marker using a laser display device with a patient-based coordinate system outside the subject to identify the position and direction of the second indicating marker.

10. A method for providing position and direction information of an electrode array for electrical stimulation therapy of the subject comprising:
mounting a plurality of first indicating markers for indicating a center position and a reference direction of the electrode array on the surface of a subject at predetermined intervals;
attaching a plurality of image markers to the surface of the subject in alignment with the center position and the reference direction of the plurality of first indicating markers;
acquiring a medical image of the subject to which the plurality of image markers is attached;
providing the acquired medical image to an external treatment planning system;
receiving a proposed position and a proposed direction of the electrode array based on the image marker from the external treatment planning system; and
mounting a second indicating marker on the surface of the subject in alignment with the proposed position and the proposed direction.

11. An apparatus 1000 for providing position and direction information of an electrode array for electrical stimulation therapy of the subject comprising:
an indicating marker unit 1100 mounting a first indicating marker for indicating a center position and a reference direction of the electrode array on a surface of a subject;
an image marker unit 1200 attaching an image marker to the surface of the subject in alignment with the center position and the reference direction of the first indicating marker 1220;
a proposed position and direction determination unit 1300 recording a medical image of the subject to which the image marker is attached, providing the acquired medical image to an external treatment planning system and receiving a proposed position and a proposed direction of the electrode array based on the image marker from the external treatment planning system;
wherein the indicating marker unit 1100 mounts a second indicating marker 1140 on the surface of the subject in alignment with the proposed position and the proposed direction based on the image marker provided from the proposed position and direction determination unit 1300.

12. The apparatus of claim 11, wherein the electrode array comprises at least one individual electrode.

13. The apparatus of claim 11, wherein the indicating marker unit 1100 further mounts a third boundary indicating marker on the surface of the subject to mark a boundary for the surface area of the subject that should not be used for the electrical stimulation therapy.

14. The apparatus of claim 11, wherein the number of the first indicating marker is equal to or greater than the number of the image markers.

15. The apparatus of claim 11, wherein the first and second indicating markers are formed by at least one method of Temporary Tattoo, Henna, Pigment, Sticker and Skin Marker.

16. The apparatus of claim 11, wherein the medical image includes at least one of CT, MRI, PET, SPECT and FMRI (Functional MRI), and
the image marker 1220 is formed of a marker that can be read in the medical image.

17. The apparatus of claim 11, wherein the electrical stimulation therapy is at least one of Tumor Treating Fields, Transcranial Direct Current Stimulation, Transcranial Alternating Current Stimulation, Transcranial Magnetic Stimulation and Radio-frequency Hyperthermia.

18. The apparatus of claim 11, wherein the mounting of the second indicating marker 1140 on the surface of the subject in alignment with the proposed position and the proposed direction based on the image marker provided from the proposed position and direction determination unit 1300 is to follow the projection line projected onto the surface of the subject.

19. The apparatus of claim 18, wherein the mounting of the second indicating marker 1140 on the surface of the subject in alignment with the proposed position and the proposed direction based on the image marker provided from the proposed position and direction determination unit 1300 performs translation and rotation from the first indicating marker using a laser display device with a patient-based coordinate system outside the subject to identify the position and direction of the second indicating marker.

20. An apparatus 1000 of providing position and direction information of an electrode array with at least one individual electrode for electrical stimulation therapy of the subject comprising:
an indicating marker unit 1100 mounting a plurality of first indicating markers for indicating a center position and a reference direction of the electrode array on the surface of the subject at predetermined intervals;
an image marker unit 1200 attaching a plurality of image markers on the surface of the subject in alignment with the center positions and the reference directions of the plurality of first indicating markers; and
a proposed position and direction determination unit 1300 recording a medical image of the subject on which the plurality of image markers is attached, providing the recorded medical image to an external treatment planning system and receiving a proposed position and a proposed direction of the electrode array based on the plurality of image markers from the external treatment planning system,
wherein the indicating marker unit 1100 mounts a second indicating marker 1140 on the surface of the subject in alignment with the proposed position and the proposed direction based on the plurality of image markers provided from the proposed position and direction determination unit 1300.
